(19) United States
(12) Patent Application Publication
(10) Pub. No.: US 2017/0367621 A1
(43) Pub. Date: Dec. 28, 2017

(54) Phlebostatic Axis Locating Device

(71) Applicants: Hung Nghiem, Los Angeles, CA (US); Khang Nghiem, Los Angeles, CA (US)

(72) Inventors: Hung Nghiem, Los Angeles, CA (US); Khang Nghiem, Los Angeles, CA (US)

(21) Appl. No.: 15/191,659

(22) Filed: Jun. 24, 2016

(51) Int. Cl. A61B 5/107; B43L 7/00

(52) U.S. Cl. CPC A61B 5/1072; B43L 7/00

(57) ABSTRACT

A phlebostatic axis locating device for locating a phlebostatic axis of a homosapien includes a straightedge and a measuring stick. The measuring stick is slidably coupled to the straightedge proximate to a terminus of the straightedge, defining an intersection. The measuring stick extends perpendicularly from the straightedge. The straightedge is configured to position on the fourth intercostal space of a substantially supine homosapien supported on a surface, such that the straightedge is substantially parallel to the surface. The straightedge extends substantially perpendicularly from a centerline of a chest of the homosapien such that the terminus of the straightedge is positioned beyond the chest. An end of the measuring stick is configured to position on the surface, with the measuring stick substantially perpendicular to the surface. A midpoint of a distance between the intersection and the surface is determined. The midpoint corresponds to a phlebostatic axis of the homosapien.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIE THE OFFICE ELECTRONIC FILING SYSTEM.

Not Applicable

### STATEMENT REGARDING PRIOR DISCLOSURES BY THE INVENTOR OR JOINT INVENTOR Not Applicable

### BACKGROUND OF THE INVENTION

### (1) Field of the Invention

### (2) Description of Related Art including information disclosed under 37 CFR 1.97 and 1.98

The disclosure and prior art relates to locating devices and more particularly pertains to a new locating device for locating a phlebostatic axis of a homosapien.

### BRIEF SUMMARY OF THE INVENTION

An embodiment of the disclosure meets the needs presented above by generally comprising a straightedge and a measuring stick. The measuring stick is slidably coupled to the straightedge proximate to a terminus of the straightedge, defining an intersection. The measuring stick extends perpendicularly from the straightedge. The straightedge is configured to position on the fourth intercostal space of a substantially supine homosapien supported on a surface, such that the straightedge is substantially parallel to the surface. The straightedge extends substantially perpendicularly from a centerline of a chest of the homosapien such that the terminus of the straightedge is positioned beyond the chest. An end of the measuring stick is configured to position on the surface, with the measuring stick substantially perpendicular to the surface. A midpoint of a distance between the intersection and the surface is determined. The midpoint corresponds to a phlebostatic axis of the homosapien.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a phlebostatic axis locating device according to an embodiment of the disclosure.

FIG. 2 is an in-use view of an embodiment of the disclosure.

FIG. 3 is a planar view of an embodiment of the disclosure.

FIG. 4 is a planar view of an embodiment of the disclosure.

FIG. 5 is a front view of an embodiment of the disclosure.

### DETAILED DESCRIPTION OF THE INVENTION

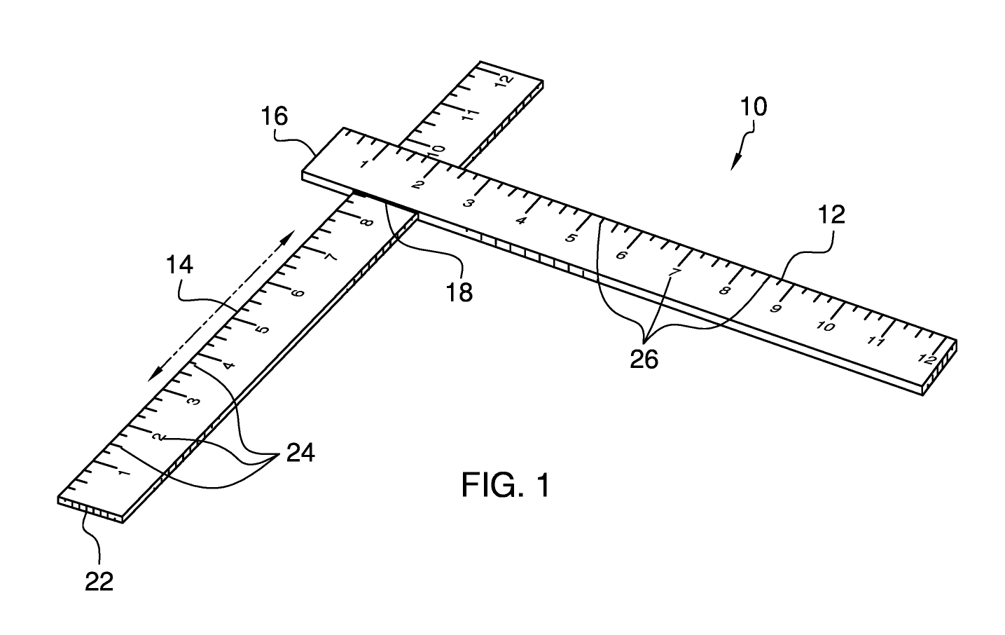

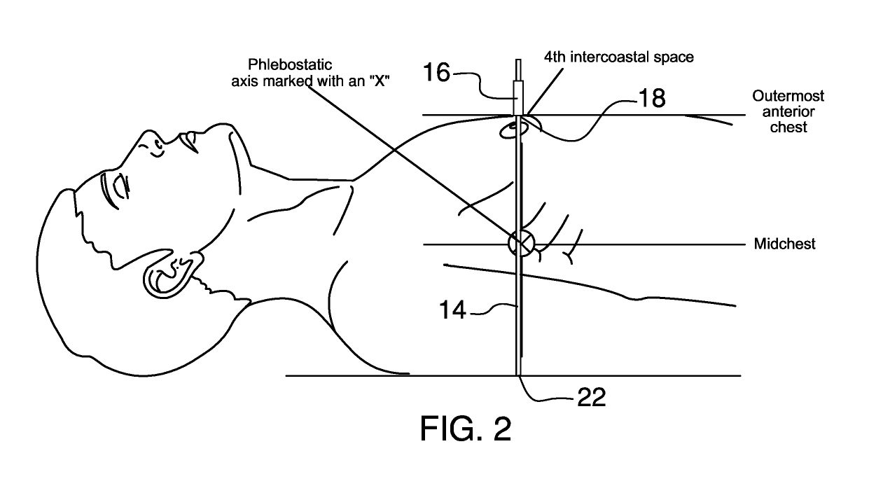

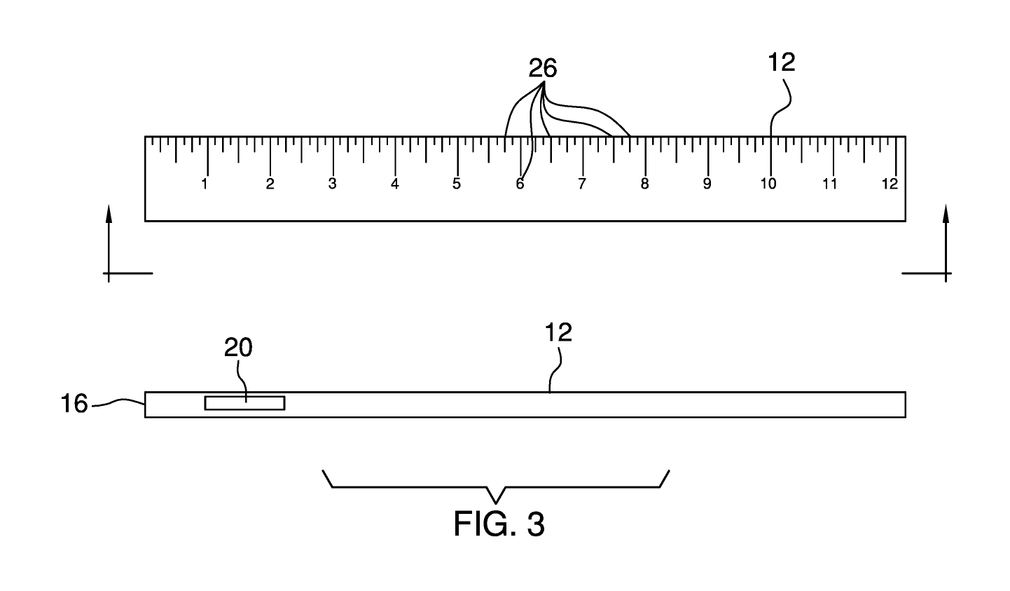

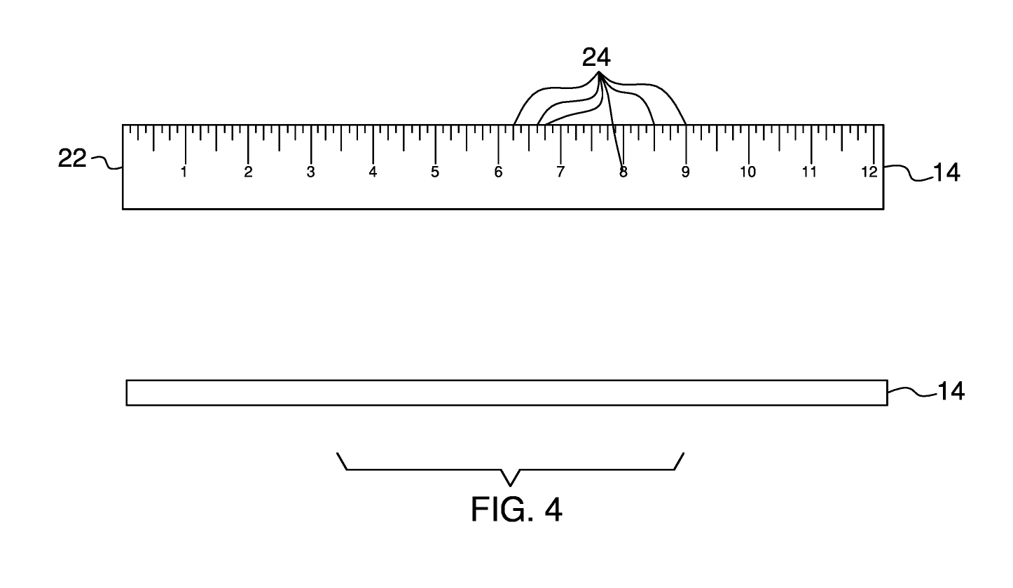

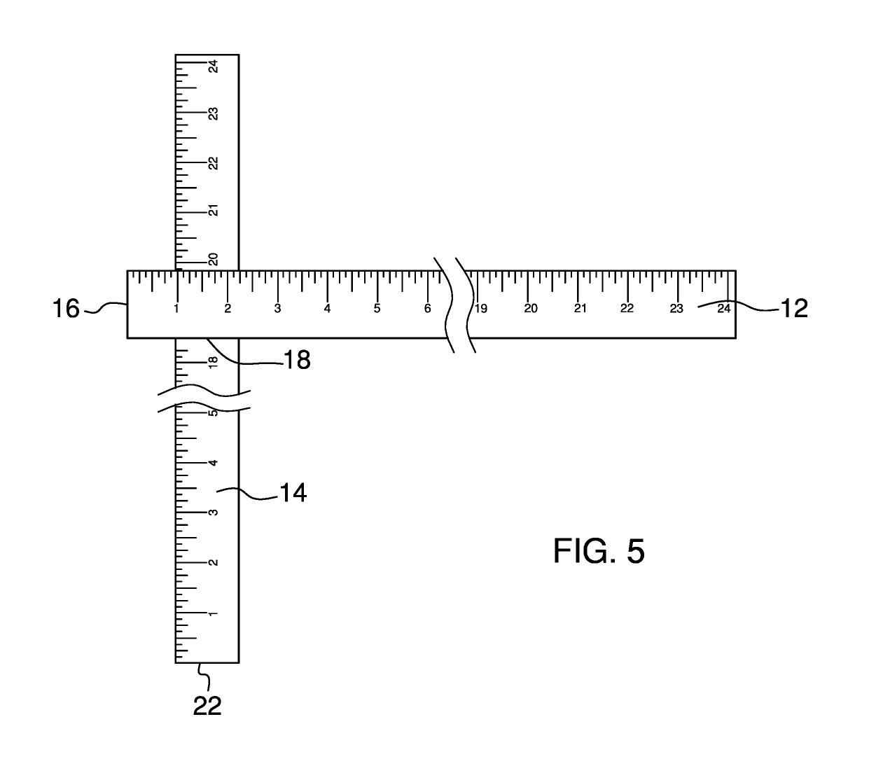

With reference now to the drawings, and in particular to FIGS. 1 through 5 thereof, a new locating device embodying the principles and concepts of an embodiment of the disclosure and generally designated by the reference numeral 10 will be described.

As best illustrated in FIGS. 1 through 5, the phlebostatic axis locating device 10 generally comprises a straightedge 12 and a measuring stick 14. The measuring stick 14 is slidably coupled to the straightedge 12 proximate to a terminus 16 of the straightedge 12, defining an intersection 18. The measuring stick 14 extends perpendicularly from the straightedge 12.

A slot 20 is positioned through the straightedge 12 proximate to the terminus 16. The slot 20 is substantially complementary to an end 22 of the measuring stick 14. The slot 20 is positioned for insertion of the end 22 of the measuring stick 14, such that the measuring stick 14 is slidably positionable within the slot 20.

In one embodiment, first indicia 24 are coupled to the measuring stick 14. The first indicia 24 are distributed longitudinally on the measuring stick 14. The first indicia 24 are evenly spaced, such that the first indicia 24 are configured to measure distances between points.

In another embodiment, second indicia 26 are coupled to the straightedge 12. The second indicia 26 are distributed longitudinally on the straightedge 12. The second indicia 26 are evenly spaced such that the second indicia 26 are configured to measure distances between points.

In one embodiment, the straightedge 12 and the measuring stick 14 comprises metal. In another embodiment, the straightedge 12 and the measuring stick 14 comprises stainless steel. In yet another embodiment, the straightedge 12 and the measuring stick 14 comprises plastic.

In use, the straightedge 12 is configured to position on the fourth intercostal space of a substantially supine homosapien. The straightedge 12 is positioned substantially parallel to a surface supporting the homosapien and extends substantially perpendicularly from a centerline of a chest of the homosapien. The terminus 16 of the straightedge 12 is positioned beyond the chest. The measuring stick 14 is slidably coupled to the straightedge 12 such that an end 22 of the measuring stick 14 is configured to position on the surface supporting the homosapien. The measuring stick 14 is positioned substantially perpendicularly to the surface. A midpoint of a distance between the intersection 18 and the surface is determined. The midpoint corresponds to a phlebostatic axis of the homosapien.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A phlebostatic axis locating device comprising:
a straightedge;
a measuring stick slidably coupled to said straightedge proximate to a terminus of said straightedge defining an intersection, said measuring stick extending perpendicularly from said straightedge; and
wherein said straightedge is configured to position on the fourth intercostal space of a substantially supine homosapien, such that said straightedge is substantially parallel to a surface supporting the homosapien and wherein said straightedge extends substantially perpendicularly from a centerline of a chest of the homosapien such that said terminus of said straightedge is positioned beyond the chest, wherein said measuring stick is slidably coupled to said straightedge such that an end of said measuring stick is configured to position on the surface supporting the homosapien and wherein said measuring stick is substantially perpendicular to the surface, such that a midpoint of a distance between said intersection and the surface is determined and wherein the midpoint corresponds to a phlebostatic axis of the homosapien.

2. The device of claim 1, further including a slot positioned through said straightedge proximate to said terminus, said slot being substantially complementary to said end of said measuring stick, wherein said slot is positioned for insertion of said end of said measuring stick such that said measuring stick is slidably positionable within said slot.

3. The device of claim 1, further including first indicia coupled to said measuring stick, said first indicia being distributed longitudinally on said measuring stick, said first indicia being evenly spaced such that said first indicia are configured to measure distances between points.

4. The device of claim 1, further including second indicia coupled to said straightedge, said second indicia being distributed longitudinally on said straightedge, said second indicia being evenly spaced such that said second indicia are configured to measure distances between points.

5. The device of claim 1, further including said straightedge and said measuring stick comprising metal.

6. The device of claim 5, further including said straightedge and said measuring stick comprising stainless steel.

7. The device of claim 1, further including said straightedge and said measuring stick comprising plastic.

8. A phlebostatic axis locating device comprising:
a straightedge;
a measuring stick slidably coupled to said straightedge proximate to a terminus of said straightedge defining an intersection, said measuring stick extending perpendicularly from said straightedge;
a slot positioned through said straightedge proximate to said terminus, said slot being substantially complementary to said end of said measuring stick, wherein said slot is positioned for insertion of said end of said measuring stick such that said measuring stick is slidably positionable within said slot;
first indicia coupled to said measuring stick, said first indicia being distributed longitudinally on said measuring stick, said first indicia being evenly spaced such that said first indicia are configured to measure distances between points;
second indicia coupled to said straightedge, said second indicia being distributed longitudinally on said straightedge, said second indicia being evenly spaced such that said second indicia are configured to measure distances between points;
said straightedge and said measuring stick comprising metal, said straightedge and said measuring stick comprising stainless steel, said straightedge and said measuring stick comprising plastic; and
wherein said straightedge is configured to position on the fourth intercostal space of a substantially supine homosapien, such that said straightedge is substantially parallel to a surface supporting the homosapien and wherein said straightedge extends substantially perpendicularly from a centerline of a chest of the homosapien such that said terminus of said straightedge is positioned beyond the chest, wherein said measuring stick is slidably coupled to said straightedge such that an end of said measuring stick is configured to position on the surface supporting the homosapien and wherein said measuring stick is substantially perpendicular to the surface, such that a midpoint of a distance between said intersection and the surface is determined and wherein the midpoint corresponds to a phlebostatic axis of the homosapien.
